(19) United States
(12) Patent Application Publication
(10) Pub. No.: US 2013/0066158 A1
(43) Pub. Date: Mar. 14, 2013

(54) Fabric Retraction Device and Method for Minimally Invasive Surgery

(71) Applicant: Carlos Rodriguez, Punta Gorda, FL (US)

(72) Inventor: Carlos Rodriguez, Punta Gorda, FL (US)

(21) Appl. No.: 13/565,560

(22) Filed: Aug. 2, 2012

Related U.S. Application Data
(60) Provisional application No. 61/515,598, filed on Aug. 5, 2011.

(51) Int. Cl. A61B 1/32

(52) U.S. Cl. 600/208

(57) ABSTRACT

The present invention is directed to methods and instruments for performing tissue retraction in minimally invasive spinal surgery. Another specific application includes surgical methods performed through the percutaneously retracted tissue at any location in a patient's body. The tissue retraction is provided by an elongated sleeve formed from fabric materials, which may include the ability to stretch beyond their original length along at least one axis, for passage of instruments or implants.

### CROSS REFERENCE TO RELATED APPLICATIONS

This application claims the benefit under 35 U.S.C. 119(e) of U.S. Application No. 61/515,598, filed Aug. 5, 2011, entitled, “FABRIC RETRACTION DEVICE AND METHOD FOR MINIMALLY INVASIVE SURGERY”, the contents of which are incorporated herein in their entirety.

### FIELD OF INVENTION

The present invention relates to instruments and methods for performing tissue retraction and surgeries through retracted tissue in minimally invasive procedures. More specifically, the present invention relates to a fabric retraction device for limiting the danger of damage to vital intervening tissues.

### BACKGROUND INFORMATION

A normal human spine is segmented with seven cervical, twelve thoracic and five lumbar segments. The lumbar portion of the spine resides on the sacrum, which is attached to the pelvis. The pelvis is supported by the hips and leg bones. The bony vertebral bodies of the spine are separated by intervertebral discs, which reside sandwiched between the vertebral bodies and operate as joints, allowing known degrees of flexion, extension, lateral bending and axial rotation.

The intervertebral disc primarily serves as a mechanical cushion between adjacent vertebral bodies, and permits controlled motions within vertebral segments of the axial skeleton. The disc is a multi-element system, having three basic components: the nucleus pulposus (“nucleus”), the anulus fibrosus (“anulus”) and two vertebral end plates. The end plates are made of thin cartilage overlying a thin layer of hard, cortical bone that attaches to the spongy, richly vascular, cancellous bone of the vertebral body. The plates thereby operate to attach adjacent vertebrae to the disc. In other words, a transitional zone is created by the end plates between the malleable disc and the bony vertebrae. The anulus of the disc forms the disc perimeter, and is a tough, outer fibrous ring that binds adjacent vertebrae together. The fiber layers of the anulus include fifteen to twenty overlapping plies, which are inserted into the superior and inferior vertebral bodies at roughly a 40-degree angle in both directions. This causes bi-directional torsional resistance, as about half of the angulated fibers will tighten when the vertebrae rotate in either direction. It is common practice to remove a spinal disc in cases of spinal disc deterioration, disease or spinal injury. The discs sometimes become diseased or damaged such that the intervertebral separation is reduced. Such events cause the height of the disc nucleus to decrease, which in turn causes the anulus to buckle in areas where the laminated plies are loosely bonded. As the overlapping laminated plies of the anulus begin to buckle and separate, either circumferential or radial anular tears may occur. Such disruption to the natural intervertebral separation produces pain, which can be alleviated by removal of the disc and maintenance of the natural separation distance. In cases of chronic back pain resulting from a degenerated or herniated disc, removal of the disc becomes medically necessary.

In some cases, the damaged disc may be replaced with a disc prosthesis intended to duplicate the function of the natural spinal disc. In other cases it is desired to fuse the adjacent vertebrae together after removal of the disc, sometimes referred to as “intervertebral fusion” or “interbody fusion.”

In cases of intervertebral fusion, it is known to position a spacer centrally within the space where the spinal disc once resided, or to position multiple spacers within that space. Procedures are known for insertion of the spacer(s) via anterior, posterior or lateral methods.

While the occurrence of successful spinal surgeries of any of the variety mentioned above has greatly improved in recent years, there continue to be challenges and room for improvement in the area of intervertebral spacers and prosthetics. In particular, a patient's precise anatomy is often not known prior to surgery, although general predictions will be available. A significant danger of performing intervertebral operations or accessing an intervertebral space during spine surgery is that of inadvertently contacting or damaging the para-spinal nerves, including the exiting nerve roots, traversing nerves and the nerves of the cauda equina. The exact location of these para-spinal nerves cannot be determined prior to the commencement of surgery. Moreover, intervertebral spaces in the spine have other sensitive nerves disposed at locations, which are not entirely predictable prior to insertion of the surgical tool into the intervertebral area. Accordingly, the danger of pinching or damaging spinal nerves when accessing an intervertebral space has proven to be quite limiting to the methods and devices used during minimally invasive spinal surgery. In addition, as cannulae are received through the patient's back, such as when performing minimally invasive spinal surgery, minor blood vessels are ruptured; thereby blocking the surgeon's vision inside the intervertebral region after the cannula has been inserted.

Additionally, while surgery is a well-planned process, not all conditions can be known beforehand and some variations will likely not be ideal. Accordingly, during surgery a surgeon will likely need to make decisions that balance speed, safety, and efficacy. One such decision can relate to the approach angle at which the spacer is inserted into the patient's body. This angle can vary depending on the surgical conditions encountered.

Still yet, the devices that are implanted during these procedures are generally not shaped to be easily insertable into the affected disc space through the narrow opening that is available with percutaneous procedures. Thus, additional retraction of the nerve roots may be required to gain insertion of the device. Such retraction can cause temporary or permanent nerve damage.

Therefore, there exists a need for minimally invasive spinal and neurosurgical surgical instruments and methods that provide access to locations within the body that eliminate or limit the danger of damage to vital intervening tissues.

### SUMMARY OF THE INVENTION

Briefly, the present invention is directed to methods and instruments for performing tissue retraction in minimally invasive spinal surgery. Another specific application includes surgical methods performed through the percutaneously retracted tissue at any location in a patient's body. The tissue retraction is provided by an elongated sleeve formed from fabric materials, which may include the ability to stretch beyond their original length along at least one axis, for passage of instruments or implants.

This construction provides multiple advantages over the prior art. First, it provides a flexible channel to the surgical site. Second, it protects intervening tissue and nerves from impingement. Third, it provides a barrier to reduce body fluids from entering the surgical site. Finally, the construction provides a woven elastic channel to the surgical site.

Accordingly, it is an objective of the present invention to provide an instrument for performing tissue retraction in minimally invasive spinal surgery.

It is a further objective of the present invention to provide a method for performing tissue retraction in minimally invasive spinal surgery.

It is yet a further objective of the present invention to provide a tissue retractor formed from fabric materials.

It is another objective of the instant invention to provide an expandable tissue retractor formed of fabric materials.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 is a top perspective view of one embodiment of the instant invention, illustrating the device in its second deployed position;

FIG. 2 is a section view taken along lines 2-2 of FIG. 1, illustrating a radio opaque filament extending through the device;

FIG. 3 is a section view taken along lines 3-3 of FIG. 1, illustrating a securing hook of the instant invention;

FIG. 4 is a top perspective view of one embodiment of the instant invention, illustrating the device in a first non-deployed position wrapped about an insertion probe;

FIG. 5 is a top perspective view of one embodiment of the instant invention, illustrating the device in a first non-deployed position wrapped and inserted into an insertion probe;

FIG. 6 is a section view taken along lines 6-6 of FIG. 1, illustrating one embodiment of the device in cooperation with a retention frame;

FIG. 7 is a perspective view of one embodiment of a retention frame of the instant invention;

FIG. 8 is a perspective view of one embodiment of a retention ring of the instant invention;

FIG. 9 is a perspective view of one embodiment of a retention ring of the instant invention;

FIG. 10 is a perspective view of one embodiment of a retention ring of the instant invention;

FIG. 11 is a perspective view of one embodiment of a retention ring of the instant invention.

### DETAILED DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated.

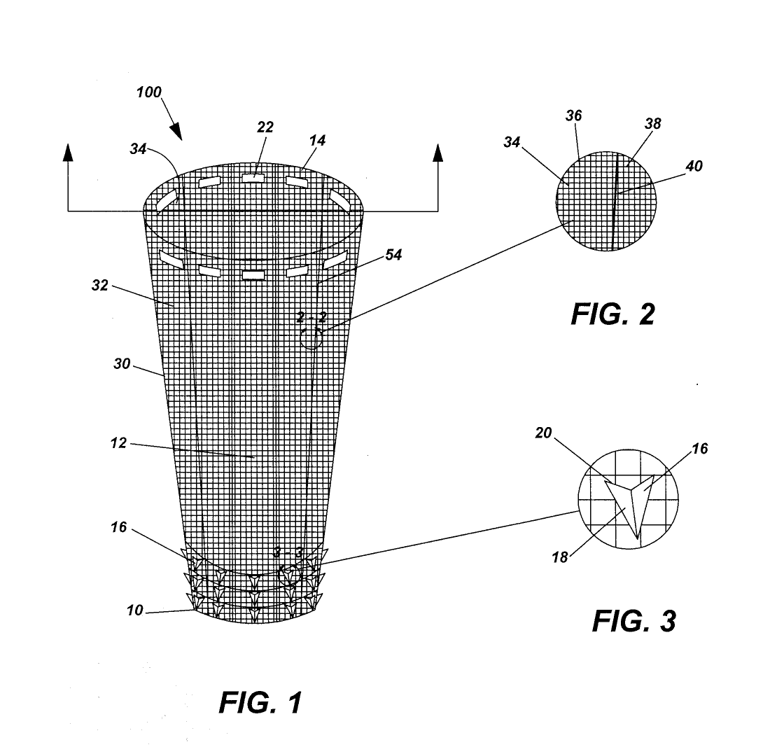

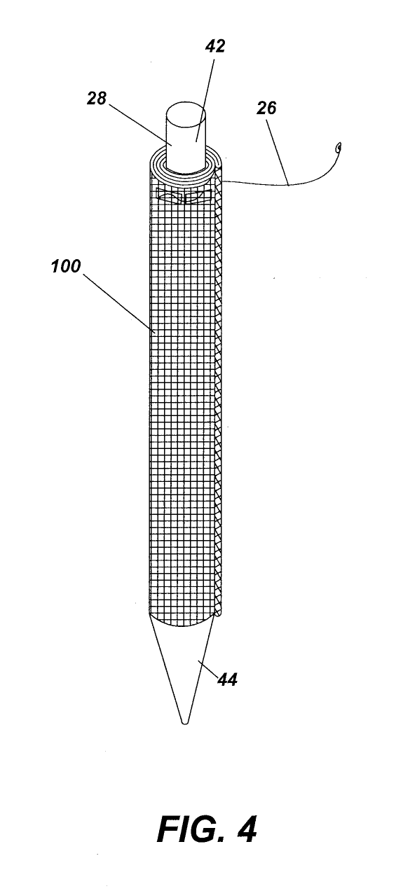

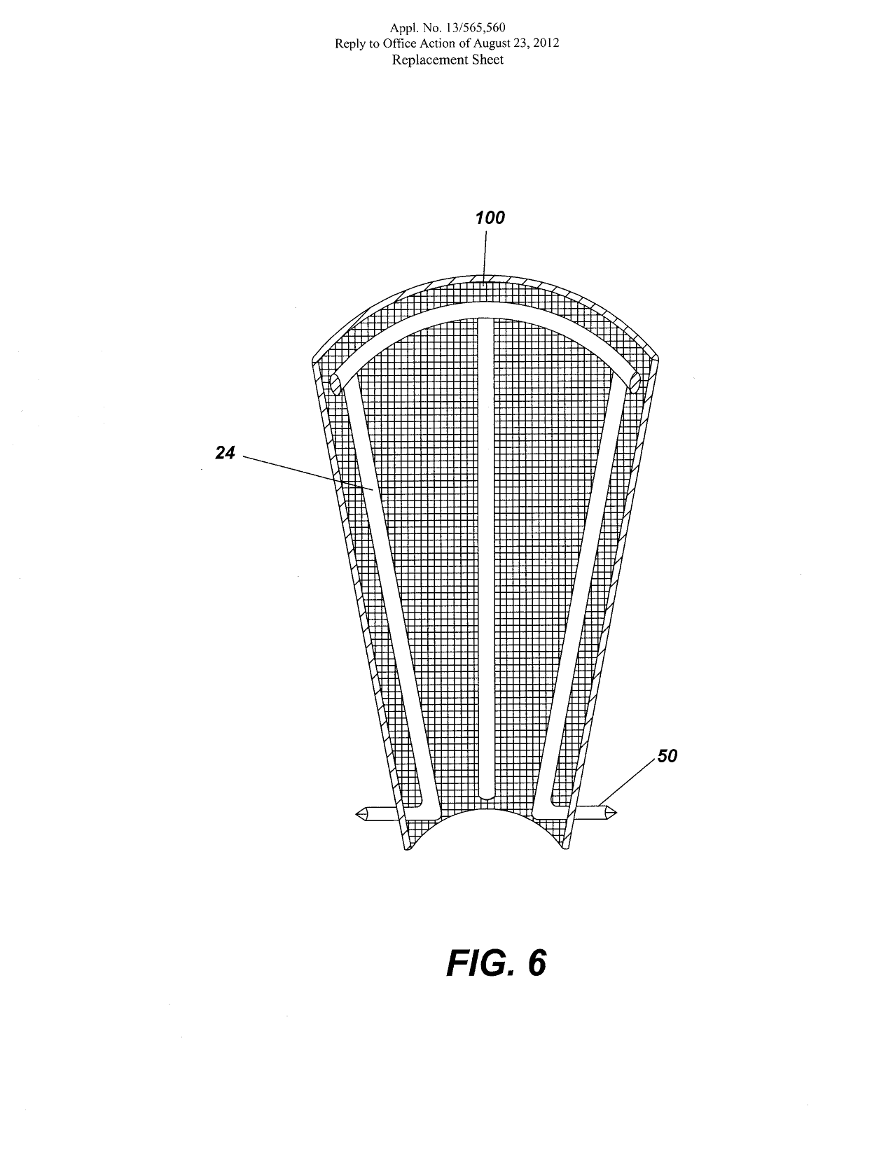

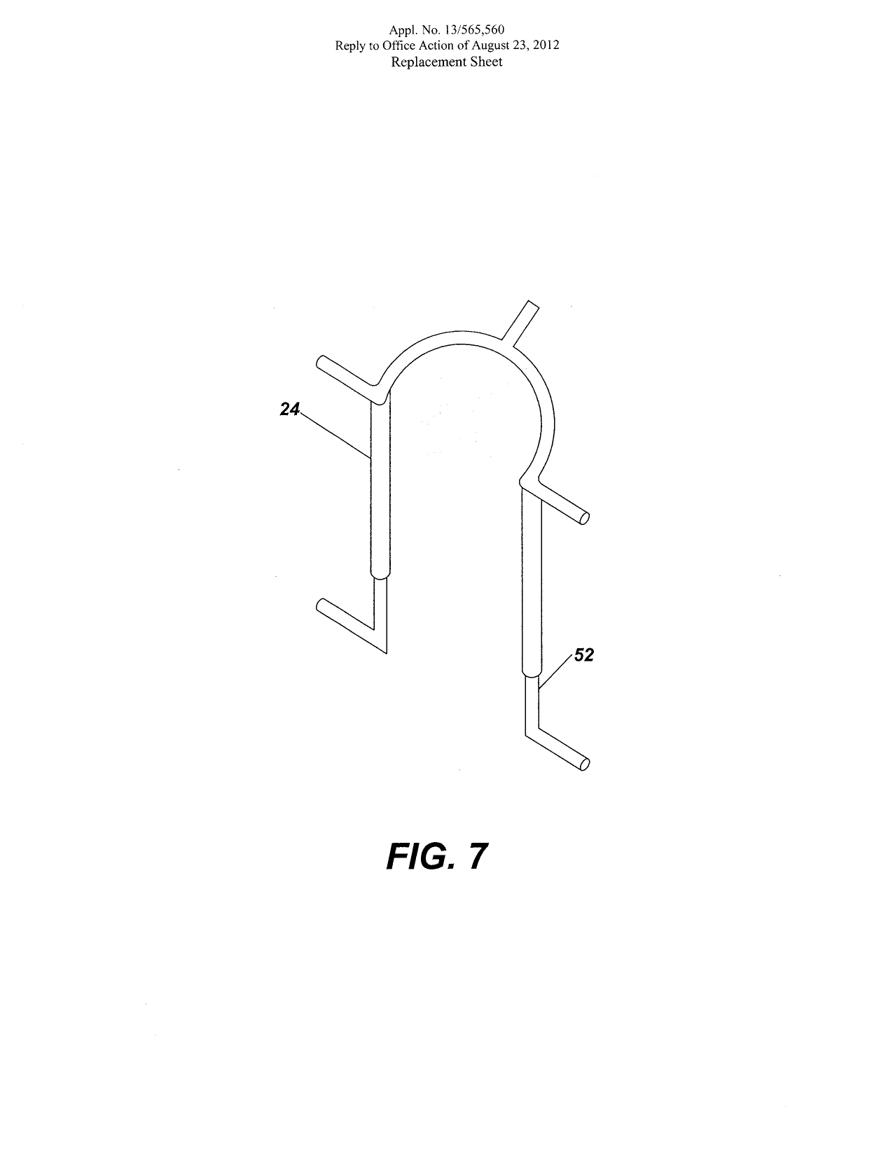

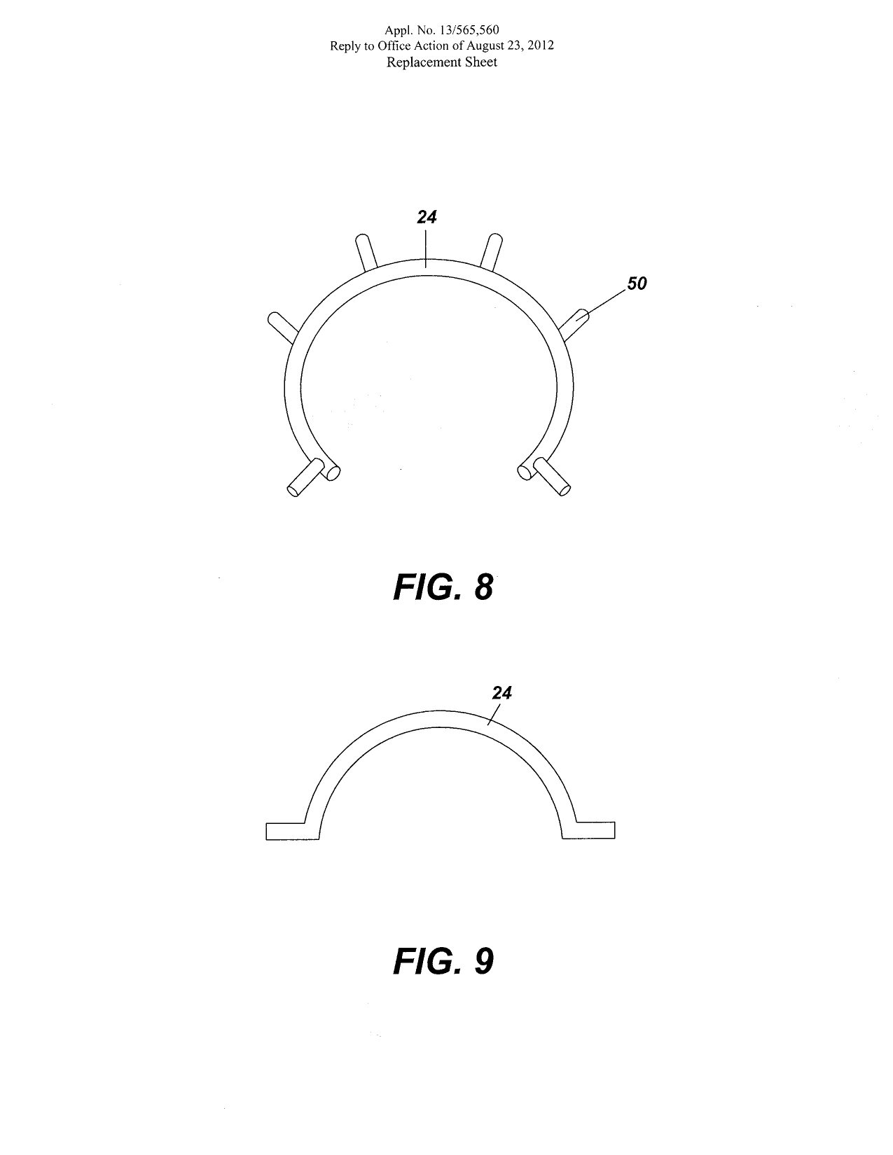

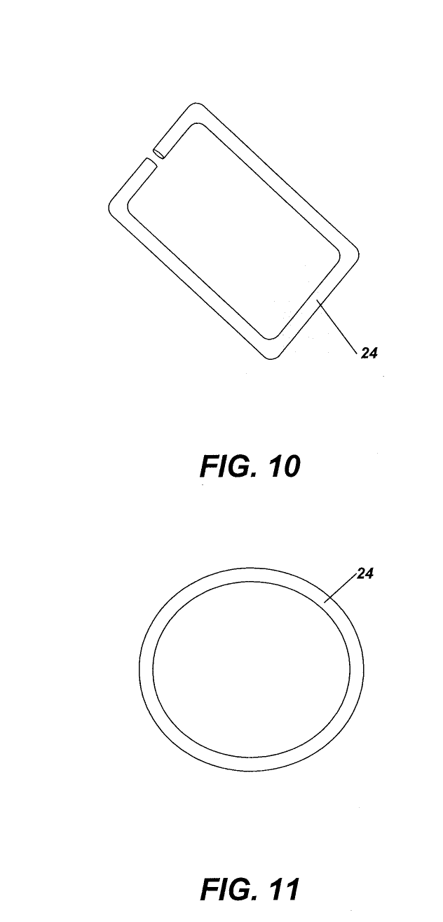

Referring generally to FIGS. 1-3, a woven retractor sleeve 100 for providing access to a surgical site is illustrated. The woven retractor sleeve 100 is constructed to include a central portion 12, a base end 10 and a proximal portion 14. The retractor sleeve is generally tubular in cross sectional shape and can be made in a variety of sizes for different types of percutaneous surgical procedures. The base portion 10 is preferably provided with at least one, and more preferably a plurality of anchor members 16. In general, the anchor members include a sloped side 18 and a catch side 20. The anchor members are preferably constructed from a relatively rigid or spring like material to allow the directional engagement of the sleeve through the tissue or bone surrounding the surgical site while the anchors engage tissue and/or bone to secure the sleeve at the desired location. However, it should also be noted that the anchor members may be constructed from the same or similar material as the sleeve without departing from the scope of the invention. In this manner, the sleeve can be incrementally advanced to the surgical site. Alternatively, the sleeve may be moved to the desired location and the anchors may be directly engaged to the tissue surrounding the surgical site. In one non-limiting embodiment, the anchor members 16 each include a V-shaped cross sectional shape, a sloped side 18 and a catch side 20. The anchor members 16 may be secured at various locations along the retractor sleeve 100 by stitching, adhesive, weaving, fasteners or any suitable combination thereof. The proximal portion 14 of the retractor sleeve is preferably larger in diameter than the base end and may include at least one, and more preferably a plurality of apertures 22 sized and shaped to cooperate with one or more cage members 24 (FIGS. 6-11). The apertures may also be utilized in conjunction with a drawstring member 26 (FIG. 4) to secure the retractor sleeve 100 to an insertion probe 28 as shown in FIG. 4.

Still referring to FIGS. 1-3. The sidewall 30 of the retractor sleeve 100 is constructed from a fabric, e.g. woven and/or knit material, to provide properties not found within rigid retraction devices. It will be appreciated by those skilled in the art that, in the fabric produced in the weaving or knitting process, the longitudinal fibers 36 and the circumferential fibers 38 may not be distributed in discrete layers in the fabric. However, in the preferred embodiment of the instant invention, the fibers of one yarn are predominantly located on the technical front 32 of the fabric and the other yarn fibers are predominantly located on the technical back 34 of the fabric. This construction provides numerous advantages with respect to rigid type retractors. The fabric material allows the retraction sleeve to be reduced significantly in size or changed in shape for insertion. The woven construction permits the use of fibers or yarns having different properties extending through the fabric in longitudinal and circumferential directions. This will allow the retractor sleeve to expand in diameter beyond its static diameter for passage of an implant or tool providing shorter term retraction of tissue, nerve roots etc. to minimize trauma to the patient. The fabric may also include one of a variety of surface or face finishing including caustic reduction, napping, brushing, sueding, and shearing to increase surface filament count to increase surface acceptance of fluids. Alternatively or in addition to the surface finishing, the fabric may be coated with a coagulant to increase surgeon visibility of the site by limiting blood flow into the surgical site. In at least one embodiment, the technical back includes fibers that extend primarily in the longitudinal direction, e.g. longitudinal fibers 36. This construction allows implants and tools to be more easily slid across the surface of the retractor sleeve while the predominantly circumferential fibers on the outside of the sleeve provide gripping to the adjacent tissue to resist movement. The longitudinal fibers are preferably constructed from a high performance material/fabric, such as a ballistic resistant material/fabric, a high tensile strength material/fabric, nylon, an aramid material such as Kevlar, Spectra, Twaron, Dyneema, another synthetic material or any mixture of these. These materials provide some rigidity to the overall length of the retractor sleeve while resisting punctures and tears. In some embodiments, radio opaque fibers 40 extend along the length or portions of the length of the retractor sleeve for cooperation with surgical radiography to aid the surgeon in locating the device in the surgical site. The circumferential fibers 38 can be manufactured from a variety of fibers including cellulosic fibers, such as cotton, or synthetic petrochemical fibers, such as polyester. The fibers may be used in 100% form, or from blended fibers, including cellulosic fiber in combination with synthetic fibers, either in mechanical or intimate blends. Spandex (such as Lycra® manufactured by DuPont), monofilament, rubber, or elastomerics may be utilized or introduced to the fabric construct to create an expandable diameter sleeve.

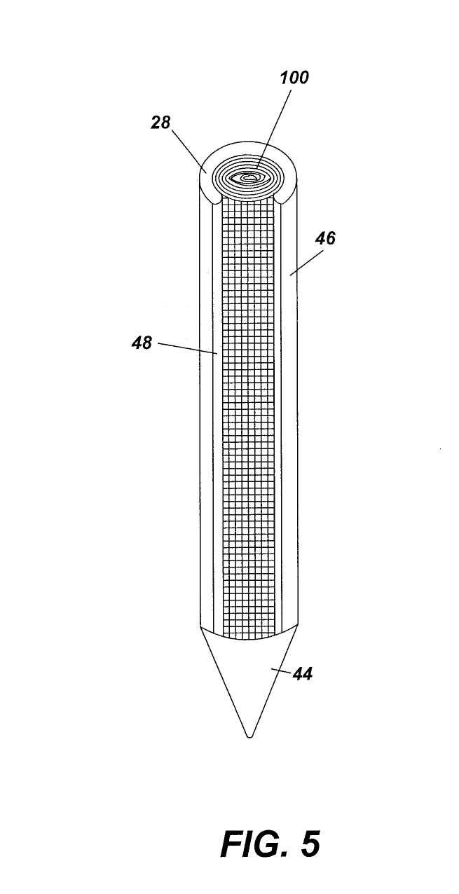

Referring to FIGS. 4 and 5, the retractor sleeve 100 is illustrated in a first compact configuration cooperating with an insertion probe 28. In FIG. 4 the retractor sleeve is wrapped about an insertion probe 28 having a central shaft 42. The central shaft includes a sharp distal end 44 for ease of tissue penetration. The insertion probe is preferably constructed of a biocompatible material and may be constructed to transfer electrical current to a neuro-monitoring device to aid the surgeon in avoiding nerve structures. A drawstring 26 may be provided to secure the retractor sleeve in place about the insertion tool. The sleeve can, after insertion be released by pulling or cutting the drawstring to release the retractor. FIG. 5 illustrates an alternative embodiment of the insertion probe 28. In this embodiment, the central shaft is a hollow shaft 46 and may include a deployment window 48 for deploying the retraction sleeve.

Referring to FIGS. 6-11, various types of cage members are illustrated. In general, the cage members are constructed and arranged to hold the retractor sleeve in a second open position. The cage members may include spikes 50 that project through the retractor sleeve to engage tissue or bone. The cage members may be constructed from various materials that may be rigid, resilient or elastic in nature. In some embodiments, the cage member may include telescoping joints 52 that may be used alone or in combination with spring and/or rigid materials.

It is to be understood that while certain forms of the invention are illustrated, it is not to be limited to the specific forms or arrangements herein described and shown.

It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A retractor sleeve for providing access to a surgical site comprising:
a sleeve assembly constructed from a fabric material to have a tubular cross section, said sleeve assembly including a base end, a central portion and a proximal portion, said base end including at least one anchor member secured to an outer surface of said fabric material, said at least one anchor member being constructed and arranged to provide engagement to tissue or bone surrounding a surgical site, whereby said base end of said sleeve assembly is securable at a desired location, said proximal portion securable outside of said surgical site, said central portion extending between said base portion and said proximal portion.

2. The retractor sleeve of claim 1 wherein said at least one anchor member includes a sloped side and a catch side to provide directional engagement to said tissue or bone surrounding the surgical site to secure the base end of said sleeve assembly at a desired location.

3. The retractor sleeve of claim 1 wherein said at least one anchor member includes a V-shaped cross sectional shape when viewed from the side of said sleeve assembly, said V-shaped anchor member having a sloped side and a catch side, said sloped side facing said base end of said sleeve assembly, said catch side facing said proximal end of said sleeve assembly.

4. The retractor sleeve of claim 2 wherein said at least one anchor member is constructed from a substantially rigid material.

5. The retractor sleeve of claim 2 wherein said at least one anchor member is constructed from a material having a spring temper.

6. The retractor sleeve of claim 1 wherein said base end includes a plurality of said anchor members.

7. The retractor sleeve of claim 1 wherein said proximal portion includes a larger diameter than said base portion.

8. The retractor sleeve of claim 1 wherein said proximal portion includes at least one aperture extending through a side portion of said sleeve assembly, said at least one aperture sized to cooperate with a cage member for holding said proximal portion of said woven sleeve in an open position.

9. The retractor sleeve of claim 8 wherein said proximal portion includes a plurality of apertures extending through a side portion of said sleeve assembly, a drawstring member interlaced through said plurality of apertures.

10. The retractor sleeve of claim 9 wherein said drawstring cooperates with an insertion probe to secure said sleeve assembly to said insertion probe in a compact configuration.

11. The retractor sleeve of claim 1 wherein said fabric sleeve is constructed using a weaving process to include a technical front formed predominantly of a first fiber and a technical back formed predominantly of a second fiber.

12. The retractor sleeve of claim 1 wherein said fabric sleeve is constructed using a knitting process to include a technical front formed predominantly of a first fiber and a technical back formed predominantly of a second fiber.

13. The retractor sleeve of claim 1 wherein said fabric is constructed to include longitudinal oriented fibers and circumferential fibers, wherein said longitudinal fibers predominantly form a technical front of said fabric and wherein said circumferential fibers predominantly form a technical back of said fabric.

14. The retractor sleeve of claim 13 wherein said technical front of said fabric forms an outer surface of said sleeve assembly and said technical back of said fabric forms an inner surface of said sleeve assembly.

15. The retractor sleeve of claim 13 wherein said technical front of said material forms an inner surface of said sleeve assembly and said technical back of said fabric forms an outer surface of said sleeve assembly.

16. The retractor sleeve of claim 13 wherein said fibers forming said technical front include a first set of material properties, and said fibers forming said technical back include a second set of material properties.

17. The retractor sleeve of claim 16 wherein said technical back includes substantial elastic properties and said technical front includes minimal elastic properties, whereby said sleeve assembly is expandable in diameter while a length of said sleeve assembly is substantially unchangeable.

18. The retractor sleeve of claim 11 wherein at least one of said technical front and said technical back include a face finishing operation to increase a surface filament count of said fabric.

19. The retractor sleeve of claim 11 wherein at least one of said technical front and said technical back include a coagulant type coating, said coagulant type coating constructed and arranged to cooperate with blood to cause coagulation thereof.

20. The retractor sleeve of claim 11 wherein at least one of said technical front and said technical back are constructed primarily of ballistic type fibers.

21. The retractor sleeve of claim 11 wherein at least one of said technical front and said technical back are constructed to include radio opaque fibers to aid the surgeon in locating the device in a surgical site.

22. The retractor sleeve of claim 1 including an insertion probe, said insertion probe including a central shaft and sharp distal end, said central shaft having a suitable length and diameter for accepting said sleeve assembly thereabout in a coiled arrangement.

23. The retractor sleeve of claim 1 including an insertion probe, said insertion probe including a tubular C-shaped central shaft and sharp distal end, said C-shaped central shaft having a suitable length and hollow inner diameter for accepting said sleeve assembly therein in a coiled arrangement, said C-shaped central shaft including a deployment window extending along the length thereof for deployment of said sleeve assembly into a surgical site.

24. The retractor sleeve of claim 22 wherein said insertion probe is constructed from an electrically conductive material.

25. The retractor sleeve of claim 1 including a cage member, said cage member constructed and arranged to cooperate with an inner surface of said sleeve assembly for holding said sleeve assembly in an open position.

26. The retractor sleeve of claim 25 wherein said cage member includes at least one spike, said spike constructed and arranged to penetrate said sleeve assembly and engage tissue surrounding said sleeve assembly.

27. The retractor sleeve of claim 25 wherein said cage is constructed from a substantially rigid material.

28. The retractor sleeve of claim 25 wherein said cage is constructed from a substantially elastic material.

29. The retractor sleeve of claim 25 wherein said cage is constructed from a shape memory material.

30. The retractor sleeve of claim 25 wherein said cage is constructed to include at least one telescoping joint.
